(19) United States
(12) Patent Application Publication
(10) Pub. No.: US 2005/0234100 A1
(43) Pub. Date: Oct. 20, 2005

(54) Insulin resistance as new action of loperamide

(75) Inventor: Juei Tang Cheng, Tainan City (TW)

Correspondence Address: BRUCE H. TROXELL, SUITE 1404, 5205 LEESBURG PIKE, FALLS CHURCH, VA, 22041, US

(73) Assignee: Yu Sheng Pharmaceutical Co., Ltd.

(21) Appl. No.: 10/826,258

(22) Filed: Apr. 19, 2004

(51) Int. Cl. A61K 31/445

(52) U.S. Cl. 514/317

(57) ABSTRACT

This application is going to claim a new action of old compound that can improve insulin resistance and this old compound named loperamide.

### BACKGROUND OF THE INVENTION

(1) Field of the Invention

The invention is going to claim a medicine that is able to improve insulin resistance and is used to improve diseases caused by insulin resistance.

(2) Description of the Prior Art

Theoretically, loperamide belonged to phenylpiperidine derivative that is generally used as the agonist of opioid μ-receptor. The phenylpiperidine derivatives, such as: meperidine and fentanyl, are addictive opioid analogues. In addition, diphenoxylate and its metabolic derivative (difenoxin) are usually combined with atropine to treat diarrhea. However, constipation is easily produced. Since loperamide is non-addictive and does not pass through blood-brain barrier, it is widely used as oral obstipantia in clinic.

[Molecular Structure] (FIG. 1)

### SUMMARY OF THE INVENTION

In clinic, loperamide is used as the medicine to treat diarrhea. In type-I diabetic rats with insufficient insulin secretion, β-endorphin is able to facilitate the utilization of glucose to result in the lowering of plasma glucose that can be eliminated by naloxone, an antagonist of opioid μ-receptors (Liu et al., 1999). Intravenous injection of loperamide, as the agonist of opioid μ-receptors, produced plasma glucose lowering effect in the STZ-induced type-I diabetic rats (Liu et al., 1999a). An activation of opioid μ-receptors seems important in the regulation of glucose homeostasis. In obese-diabetic ob/ob mice with insulin resistance, opiates were also observed to lower plasma glucose (Bailey et al., 1987). Besides, insulin resistance is easily induced in the opioid μ-receptor knock-out mice (Cheng et al., 2003) showing the relationship between opioid receptor and insulin resistance.

Therefore, it is the main object of the present invention to assure the significant improvement effect of loperamide on insulin resistance.

According to the object of the present invention, a medicine that includes loperamide to improve diseases caused by insulin resistance is proposed.

### BRIEF DESCRIPTION OF THE DRAWINGS

The present invention will now be specified with reference to its preferred embodiment illustrated in the drawings, in which FIGS. 2˜11.

FIG. 1: Molecular Structure.

FIG. 2: shows the effect of loperamide on plasma glucose (upper figure) and insulin (lower figure) levels during glucose tolerance test in fructose-induced insulin resistance of Wistar rats, wherein the abdomens of test group subjects were injected with four different doses of loperamide; ●: 2 μg/kg; ▪: 6 μg/kg; Δ: 12 μg/kg; ▴: 18 μg/kg; □ represents control group (whose abdomens were injected with same amount of vehicle). At 30 minutes later, four groups of rats were given 1 g/kg of glucose separately, which was used as the zero point, and blood samples were collected 30, 60, 90, and 120 minutes later. Data were means±SEM from each group (N=8).

FIG. 3: shows the effect of loperamide on glucose tolerance test in fructose-induced insulin resistance of Wistar rats, each column showed the area under the curves of plasma glucose (upper figure) and plasma insulin (lower figure). Data were means±SEM from each group (N=8). *P<0.05, **P<0.01, ***P<0.001, compared with the data of rats given same volume of vehicle.

FIG. 4: shows the effect of loperamide on glucose-insulin index in fructose-induced insulin resistant rats. Data were means±SEM from each group (N=8). *P<0.05, **P<0.01, ***P<0.001, compared with the data of rats given same volume of vehicle.

FIG. 5: shows the effect of opioid μ-receptor blocker on loperamide-induced change of glucose-insulin index in fructose-induced insulin resistant rats. Data were means±SEM from each group (N=8). *P<0.05, **P<0.01, ***P<0.001, compared with the data of rats given same volume of vehicle.

FIG. 6: shows the effect of loperamide on plasma glucose (upper figure) and plasma insulin (lower figure) during glucose challenge test in Zucker-diabetic fatty rats, wherein the abdomens of test group subjects (Zucker rats) were injected with three different doses of loperamide; ▪: 2 μg/kg; Δ: 6 μg/kg; ▴: 18 μg/kg; while □ or ● was the control group: including fat and lean Zucker rates (whose abdomens were injected with same amount of vehicle). 30 minutes later, five groups of animals were separately given 0.5 g/kg of glucose, which was used as the zero point, and blood samples were collected 5, 10, 20, 30, 60, 90, and 120 minutes later. Data were means±SEM from each group (N=8).

FIG. 7: shows the effect of loperamide on glucose challenge test in Zucker-diabetic fatty rats, each column showed the area under the curves of plasma glucose (upper figure) and plasma insulin (lower figure). Data were means±SEM from each group (N=6). *P<0.05, **P<0.01, ***P<0.001, compared with the data of fatty Zucker rats given same volume of vehicle.

FIG. 8: shows the effect of loperamide on glucose-insulin index in Zucker-diabetic fatty rats. Data were means±SEM from each group (N=6). *P<0.05, **P<0.01, ***P<0.001, compared with the data of fatty Zucker rats given same volume of saline (B).

FIG. 9: shows the increasing effect of loperamide on glucose uptake in nsulin resistant C₂C₁₂ myoblasts induced by TNF-α. Data were means±SEM from each group (N=8). *P<0.05, **P<0.01, ***P<0.001, compared to the data of insulin-stimulated C₂C₁₂ myoblasts (control). ##P<0.01, ###P<0.001, compared to the data of TNF-α induced insulin resistant C₂C₁₂ myoblasts receiving same volume of vehicle.

FIG. 10: shows the influence of opioid μ-receptor blocker, naloxone, to the increasing effect of loperamide on glucose uptake in insulin resistant C₂C₁₂ myoblasts induced by TNF-α. Data were means±SEM from each group (N=8). *P<0.05, **P<0.01, ***P<0.001, compared to the data of insulin-stimulated C₂C₁₂ myoblasts (control). ##P<0.01, ###P<0.001, compared to the data of TNF-α induced insulin resistant C₂C₁₂ myoblasts receiving same volume of vehicle, i.e., V-1 or V-2.

FIG. 11: shows the influence of opioid μ-receptor blocker, naloxonazine, to the increasing effect of loperamide on glucose uptake in insulin resistant C₂C₁₂ myoblasts induced by TNF-α. Data were means±SEM from each group (N=8). *P<0.05, **P<0.01, ***P<0.001, compared to the data of insulin-stimulated C₂C₁₂ myoblasts (control). ##P<0.01, ###P<0.001, compared to the data of TNF-α induced insulin resistant C₂C₁₂ myoblasts receiving same volume of vehicle, i.e., V-1 or V-2.

### REFERENCES

Bailey C J, and Flatt P R. Increased responsiveness to glucoregulatory effect of opiates in obese-diabetic ob/ob mice. Diabetologia 30: 33-37 (1987)
Baron A D, Brechtel G, Wallace P, and Edelman S V. Rates and tissue sites of non-insulin-and insulin-mediated glucose uptake in human. Am. J. Physiol. 255: E769-E774 (1988)
Carswell E A, Old L J, Kassel R L, Green S, Fiore N, and Williamson B. An endotoxin-induced serum factor that causes necrosis of tumors. Proc. Natl. Acad. Sci. U.S.A. 72: 3666-3670 (1975)
Cheng J T, Lin. I M, and Su C F. Rapid induction of insulin resistance in opioid μ-receptor knock-out mice. Neurosci. Lett., 339: 139-142 (2003)
Crist G H, Xu B. Lanoue F, Lang C H. Tissue-specific effects of in vivo adenosine receptor blocked on glucose uptake in Zucker rats. FASEB J 12: 1301-1308 (1998)
Derek L R, and Yehiel Z. Recent advances in our understanding of insulin action and insulin resistance. Diabetes Care 24: 588-597 (2001)
Erik J H, Stephen J, Tyson R K, Mary K T, and Michael K. Selective angiotensin II receptor antagonism reduces insulin resistance in obese Zucker rats. Hypertension 38: 884-890 (2001)
Greenberg A S, and Mcdaniel M L. Identifying the links between obesity, insulin resistance and β-cell function: potential role of adipocyte-derived cytokines in the pathogenesis of type 2 diabetes. Eur. J. Clin. Invest. 32: 24-34 (2002)
Hauner H, Pertuschke T, Russ M, Röhrig K, and Eckel J. Effects of tumor necrosis factor alpha (TNF-α) on glucose transport and lipid metabolism of newly-differentiated human fat cells in cell culture. Diabetologia 38: 764-771 (1995)
Hotamisligil G S, Shargill N S, and Spiegelman B M. Adipose expression of tumor necrosis factor-alpha: direct role in obesity-linked insulin resistance. Science 259: 87-91 (1993)
Hotamisligil G S, and Spiegelman B M. Tumor necrosis factor alpha: a key component of the obesity-diabetes link. Diabetes 43: 1271-1278 (1994)
Hotamisligil G S, Murray D L, and Choy L N. Spiegelman B M. Tumor necrosis factor α inhibits signaling from the insulin receptor. Proc. Natl. Acad. Sci. 91: 4854-4858 (1996)
Kara R F, Michelle S S, Tyson R K, Melanie B S, Erik B Y, and Erik J H. Effects of exercise training and ACE inhibition on insulin action in rat skeletal muscle. J. Appl. Physiol. 89: 687-694 (2000)
Liu I M, Chi T C, Chen Y C, Lu F H, and Cheng J T. Activation of opioid mu-receptor by loperamide to lower plasma glucose in streptozotocin-induced diabetic rats. Neurosci. Lett. 265: 183-186 (1999a)
Liu I M, Niu C S, Chi T C, Kuo D H, and Cheng J T. Investigation of the mechanism of the reduction of plasma glucose by cold-stress in streptozotocin-induced diabetic rats. Neurosci. 92: 1137-1142 (1999b)
Margolis R N. Hepatic glycogen synthase phosphatase and phosphorylase phosphatase activities are increased in obese (fa/fa) hyperinsulinemic Zucker rats: effects of glyburide administration. Life Sci. 41: 2615-2622 (1987)
Mikael R, Andrea D, Vanessa V H, Hans H, Martin B, Leif P, Fredrick L, and Peter A. Mapping of early signaling events in tumor necrosis factor-α-mediated lipolysis in human fat cells. J. Biol. Chem. 277: 1085-1091 (2002)
Ruan H, Hacohen N. Golub T R, Van P L, and Lodish H F. Tumor necrosis factor-alpha suppresses adipocyte-specific genes and activates expression of preadipocyte genes in 3T3-L1 adipocytes: nuclear factor-kappa B activation by TNF-alpha is obligatory. Diabetes 51: 1319-1336 (2002)
Saengsirisuwan V, Kinnick T R, Schmit M B, and Henriksen E J. Interactions of exercise training and lipoic acid on skeletal muscle glucose transport in obese Zucker rats. J. Appl. Physiol. 91: 145-153 (2001)
Smallridge R C, Kiang J G, Gist I D, Fein H G, and Gallowat R J. U-73122, an aminosteroid phospholipase C antagonist, noncompetitively inhibits thyrotropin-releasing hormone effects in GH₃ rat pituitary cell. Endocrinology 131: 1883-1888 (1992)
Stephans J M, and Pekala P H. Transcriptional repression of C/EBP-α and GLUT4 genes in 3T3-L1 adipocytes by tumor necrosis factor-α. J. Biol. Chem. 267: 13580-13584 (1992)
Wuarin L, Namdev R. Burns J G, Fei Z J, and Ishii D N. Brain insulin-like growth factor-II mRNA content is reduced in insulin-dependent and non-insulin-dependent diabetes mellitus. J. Neurochem. 67: 742-751 (1996)
Ziel F H, Venkatesan N, and Davidson M B. Glucose transport is rate limiting for skeletal muscle glucose metabolism in normal and STZ-induced diabetic rats. Diabetes 37: 885-890 (1988)

### DESCRIPTION OF THE PREFERRED EMBODIMENT

The invention disclosed herein is going to claim a medicine named loperamide that is able to improve insulin resistance. In the following description, numerous details are set forth in order to provide a thorough understanding of the present invention. It will be appreciated by one skilled in the art that variations of these specific details are possible while still achieving the results of the present invention. In other instance, well-known components are not described in detail in order not to unnecessarily obscure the present invention.

[Materials and Methods]

1. Animal Source in the Experiments

1.1 Animal Source

Male Wistar rats weighting 200-250 g were obtained from the animal center of National Cheng-Kung University, while the Zucker-diabetic fatty rats provided by Dr. K. KOMEDA (Animal Research Center, Tokyo Medical University. Tokyo, Japan) to bread in the same animal center were aged 8 weeks. They were maintained in a temperature-controlled room (25±1 □) and kept on a 12:12 light-dark cycle (light on at 06:00 h). Food and water were available ad libitum.

1.2 Dosing Method

Loperamide was dissolved in saline solution; different volumes of solution were adjusted depending on animal weight to meet the desired concentration (mg/kg) for treatment.

1.3 Examination of Insulin Resistance Improvement

Animals were food-restricted and given only water to drink for overnight before the experiment. On the morning of examination, the basal blood sample (0.1 ml) drawn from the tail veins of these rats were regarded as 0 min samples. Then, each animal was immediately received an intraperitoneal injection of glucose at 1 g/kg body wt to induce the glucose tolerance test (IGTT). Blood samples from the tail vein were drawn at 5, 10, 20, 30, 60, 90, 120 min after the glucose feeding for measurement of plasma levels of glucose and insulin. Immediately after the completion of the IGTT, all animals received 2 ml of sterile saline subcutaneously to compensate for plasma loss. The obtained whole blood was thoroughly mixed with 10 IU heparin and centrifuged at 13,000×g to separate the plasma. Concentration of plasma glucose was measured by the glucose oxidase method via an analyzer (Quik-Lab, Ames, Miles Inc., Elkhart, Ind., USA) with samples run in duplicate. Enzyme-linked immunosorbent assay was carried out to measure plasma insulin using the commercial kit (Penisula Lab. Inc., Belmont, Calif., USA). Glucose-insulin index was calculated as the product of the glucose and insulin areas under the curve (AUC) as described previously (Kara et al., 2000). In order to evaluate whether loperamide could improve insulin resistance, four different doses of loperamide (2 [symbol] 6 [symbol] 12 [symbol] 18 μg/kg) were injected into abdomen of each animal 30 minutes before the injection of glucose.

1.4 Induction of Insulin Resistance in Wistar Rats

After 2 weeks on standard chow (Purina Mills, Inc.), half of the Wistar rats were randomly assigned to receive the fructose-rich chow (Teklad, Madison, Wis.) containing 60% fructose for 4 additional weeks to induce insulin resistance that was confirmed by the loss of tolbutamide action. The other rats still received standard chow during the 4-week period.

1.5 Plasma Glucose Measurement Method

The blood sample was centrifuged to obtain plasma. Then, 10 μl of plasma was mixed with 1.0 ml of Glucose Kit Reagent (Biosystems S.A., Barcelona, Spain) at 37° C. for 10 minutes. The glucose concentration was measured in duplicate using Quik-lab analyzer (Ames, Miles Inc., Elkhart, Ind., USA) and expressed to the value of mg/dl. Results of plasma glucose lowering activity were calculated as percentage decrease of the initial value according to the formula: (Gi−Gt)/Gi×100% where Gi was the initial glucose concentration and Gt was the plasma glucose concentration after treatment of loperamide or same volume of vehicle.

1.6 Measurement of Insulin Concentration

Enzyme-linked immunosorbent assay (ELISA) was carried out to measure plasma insulin using the commercial kit (Penisula Lab. Inc., Belmont, Calif., USA). The measurement principle utilized the polyclonal antibodies in rabbit to directly identify the carboxyl-terminal of human insulin. Biotinylated peptide would compete with test subject the integration position with antibodies. After washing away the biotinylated peptides that was not integrated with antibodies, streptavidin-conjugated Horseradish Peroxidase (SA-HRP) was used to react with immobilized primary antibody/biotinylated peptide complex. Then, TMB (3,3′,5,5′-Tetramethyl Benzidine Dihydrochloride) is added to react with HRP and then yellow color was shown. The lightness or darkness of color was determined by the amount of biotinylated peptide integrated with antibodies. When the more non-biotinylated peptide was integrated with antibodies, which meant less biotinylated peptide/SA-HRP was integrated, the lighter shade of yellow it showed. The standard curve was ploted according to the amounts of light absorption of standard subjects at 450 nm. Then through log/logit, the insulin concentration was calculated using MicroReader™ 4 Plus.

2. Measurement of Glucose Uptake in Cultured C₂C₁₂ Myoblasts

2.1. C₂C₁₂ Myoblast in Culture

The C₂C₁₂ cells, obtained from Culture Collection and Research Center (CCRC 60083) of the Food Industry Institute (Hsin-Chiu City, Taiwan) were plated at 5×10⁴ cells/dish in 35-mm diameter culture dishes in Dulbecco's modified Eagle's medium (DMEM) (GIBCO BRL, Gaithersburg, Md.) supplemented with 10% fetal bovine serum (FBS) (GIBCO BRL) and 1% antibiotic solution (penicillin 10,000 U/ml, streptomycin 10 mg/ml, amphotericin B 25 μg/ml) and were grown to 80% confluence at 37° C. in humidified atmosphere containing 5% CO₂. Myoblast differentiation was induced with DMEM supplemented with 5% horse serum, L-glutamine, and penicillin/streptomycin for 72 h. Differentiated myotubes were starved for 5 h in serum-free DMEM before treatment as described previously (Sheriff et al., 1992).

2.2. Effect of Loperamide on Glucose Uptake in C₂C₁₂ Myoblasts

Glucose uptake was determined using 2-[¹⁴C]-deoxy-D-glucose (2-DG) (New England Nuclear, Boston, Mass.). After 5 h of serum starvation, cells were incubated with or without pharmacological inhibitors at indicated concentrations for 30 min at 37° C. Then, the cells were incubated with loperamide at indicated concentrations at 37° C. for another 30 min under continuous shaking at 40 cycles/min. The cells were further incubated with 2-DG (1 μCi/ml) for 5 min at 37° C. Uptake was terminated by aspiration of the solution. Cells were then washed three times, and radioactivity associated with the cells was determined by cell lysis in 1 M NaOH, and the aliquots were neutralized to be estimated in scintillation counter. 2-DG uptake was expressed as the percentage of the basal 2-DG uptake that was taken as 100% from samples incubated with DMEM only. Nonspecific uptake was obtained by parallel determinations in the presence of 20 μmol/l cytochalasin B (Sigma, St. Louis, Mo., USA).

2.3. Effect of Loperamide on Glucose Uptake in C₂C₁₂ Myoblasts with Insulin Resistance Induced by TNF-α

Cultured C₂C₁₂ myoblasts (5×10⁶) were incubated with loperamide at desired concentration or the same volume of vehicle in a cultivation condition oxygenated with a mixture of 95% O₂ and 5% CO₂ at 37° C. temperature for 3 days. Then, tumor necrosis factor-α (TNF-α) at concentration of 10 ng/ml was added. One hour later, cells were flushed down with 0.05% trypsin and put under centrifugal force at 13,000 rpm for 5 minutes, and clear liquid on top was then thrown away. Insulin (1 M) was used to simulate glucose uptake into cells using 2-[1-¹⁴C]-Deoxy-D-Glucose (2-DG) (the final concentration of isotope was 0.25 μCi/ml) as indicator for 5-min incubation. The reaction was terminated in ice-bath. Then, C₂C₁₂ myoblasts were washed with buffer liquid three times. Finally, cells were lysed with 1 M NaOH and put into scintillation vial. The 2-DG specific uptake of cells was measured as indicated above.

3. Statistical Analysis

Parametric data were expressed as the mean±s.e.m. The N in the text refers to the number of separate experiments. Multiple comparisons were analyzed by ANOVA and Dunnett's post-hoc test. The P value of 0.05 or less was considered as significant statistically.

[Results]

1. Effect of Loperamide on Insulin Resistance in Rats Received Fructose-Rich Chow

Using glucose-insulin index (Kara et al., 2000) as indicator, effect of loperamide on insulin resistance was investigated in fructose-fed rats. The control group was treated with the same volume of saline, while test groups were injected loperamide at four doses (2 [symbol] 6 [symbol] 12 [symbol] 18 μg/kg) into abdomens. In order to rule out the possibility to interaction on intestinal absorption, because loperamide can modify intestinal function, 1 g/kg glucose was intraperitoneally injected but not oral administered at 30 minutes later to induce glucose tolerance test (IGTT).

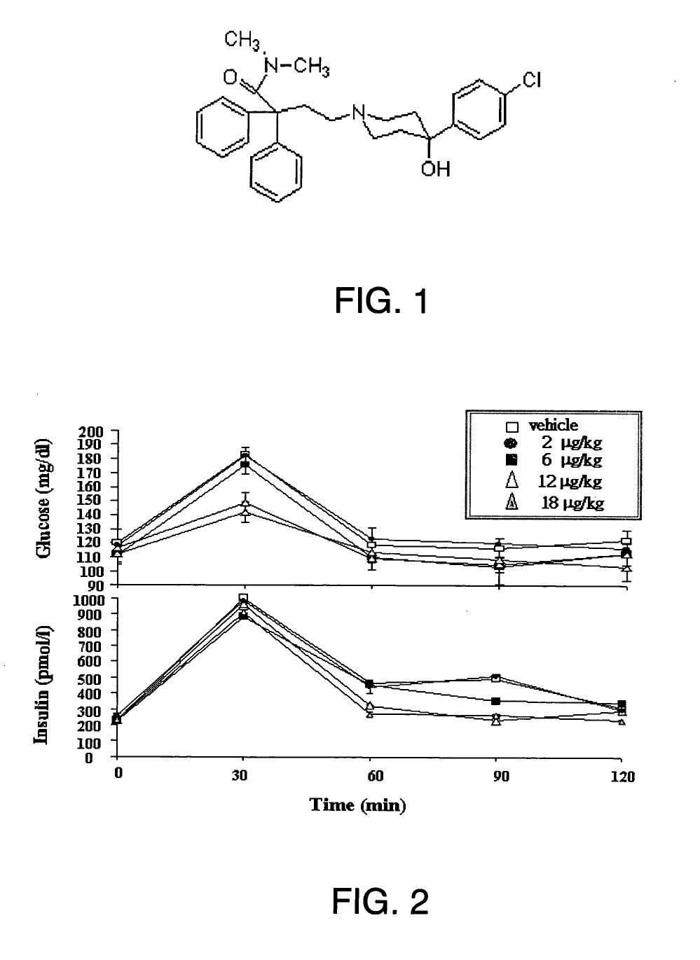

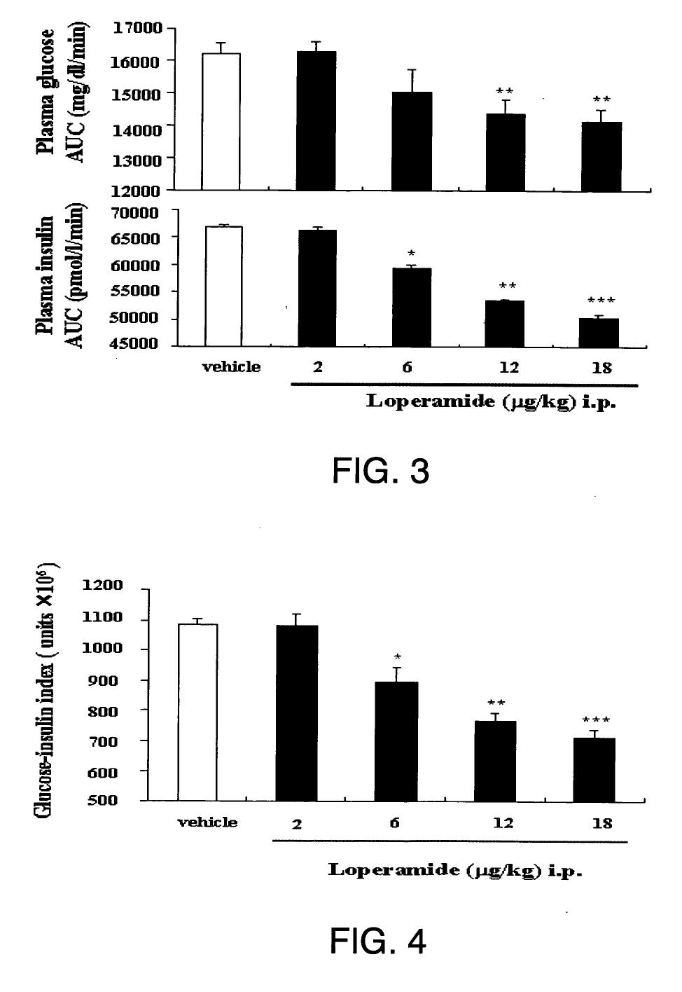

An increase of the plasma glucose and the plasma insulin during IGTT were significantly higher in the rats fed fructose-rich chow than that in regular chow-fed group (FIG. 1). Also, the AUC levels of glucose and insulin during IGTT in the fructose-rich chow-fed rats were markedly higher than that from regular chow-fed rats (FIG. 2). Then, the glucose-insulin index in the fructose-rich chow-fed rats given an oral glucose load was 12-fold of the values obtained from rats received standard chow (FIG. 3).

After injection of loperamide into the abdomens of fructose-fed rats, 60 minutes later (i.e., 30 minutes after given glucose), loperamide was found to reduce the raise of plasma glucose significantly as compared with control group and this effect was observed in a dose-dependent manner during IGTT and the total AUC for the glucose response was markedly lower than that from vehicle-treated control, but the level was still higher than that in the standard chow-fed rats. Otherwise, insulin levels in plasma from the fructose-fed rats and the incremental area under the insulin curve during IGTT were lowered by loperamide (FIG. 2). Also, loperamide reduced the value of glucose-insulin index in fructose-rich chow-fed rats during IGTT in a dose-related manner (FIG. 3).

2. Effect of Opioid μ-Receptor Blockade on Change of Glucose-Insulin Index by Loperamide.

In an attempt to know the role of opioid μ-receptors in the action of loperamide, an effective dose of naloxone at the dose (1 mg/kg) sufficient to inhibit opioid μ-receptors was injected in the same manner at 1 hour before injection of the maximum dose (18 μg/kg) of loperamide. In the presence of naloxone, the actions of loperamide to lower the value of glucose-insulin index was reversed to that near to the value in fructose-rich chow-fed rats received vehicle treatment (FIG. 4). Otherwise, naloxone at the treated dose (1 mg/kg) did not modify the value of glucose-insulin index directly.

3. Effect of Loperamide on Glucose Challenge Test in Zucker-Diabetic Fatty Rats

In order to know similar action of loperamide is also effective in genetic animal, the present study employed the Zucker-diabetic fatty rats to investigate IGTT in addition. Also, control group was treated with the same volume of vehicle (saline). The only difference is that glucose (0.5 g/kg) was given by intravenous injection at 30 minutes later of treatment.

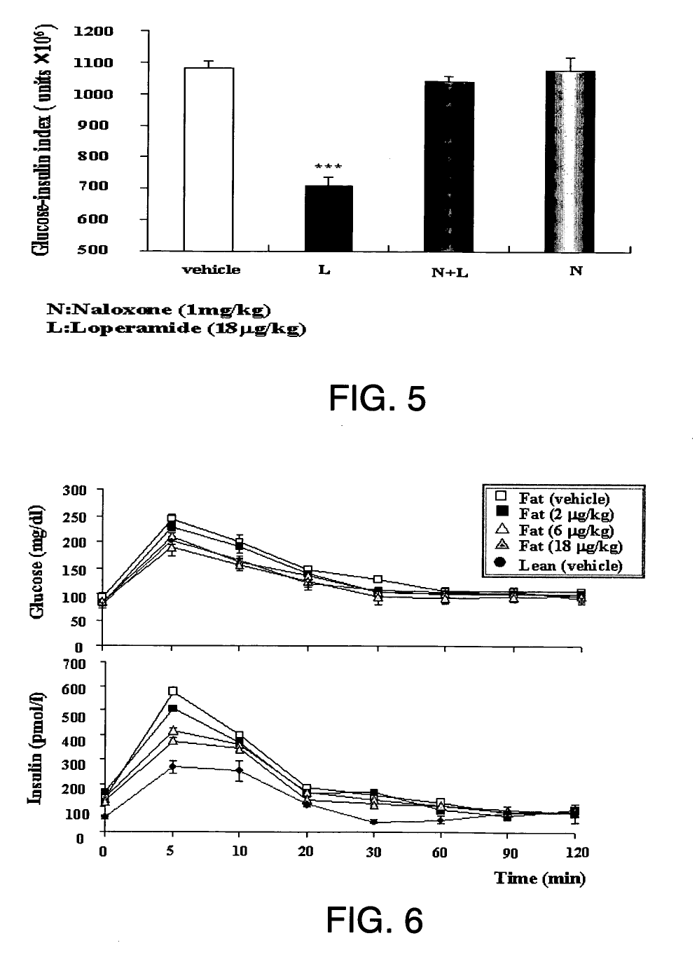

Similarly, the plasma glucose of Zucker-diabetic fatty rats in the control group treated with same volume of vehicle was found to reach its highest point 5 minutes after intravenous injection of glucose (FIG. 5); the plasma glucose raised from the basal 93.7±8.0 to 243.7±8.5 mg/dl.

After treatment with loperamide, as shown in FIG. 5, a marked of the raised plasma glucose and lowering of plasma glucose were observed in Zucker-diabetic fatty rats at 35 min later (i.e., 5 min after given glucose). The reduction of plasma insulin by loperamide was obtained in a dose-dependent fashion at 5 min later of glucose injection (FIG. 5).

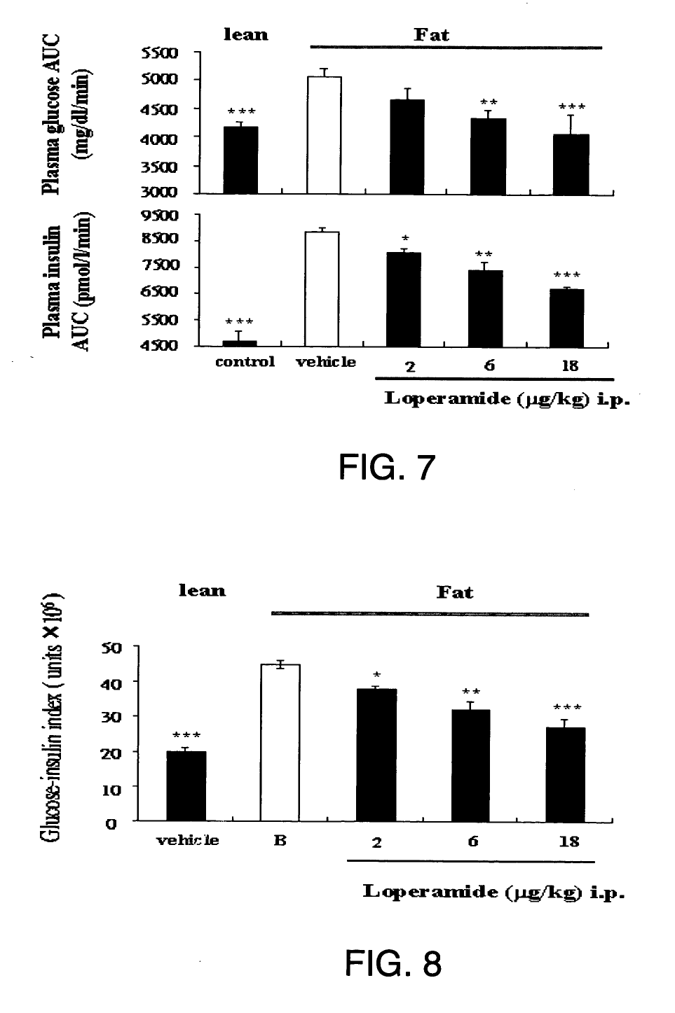

The total area (AUC) of plasma insulin in was reduced by loperamide in a dose-dependent manner (FIG. 6). Also, the AUC of plasma glucose was inhibited by loperamide but at a higher dosing. Otherwise, Zucker-diabetic fatty rats showed the value of glucose-insulin index in a way markedly higher than the lean control. Also, lopeamide dose-dependently decreased the value of glucose-insulin index in Zucker-diabetic fatty rats (FIG. 7), indicting that loperamide had the same ability to improve the insulin resistance in Zucker-diabetic fatty rats.

4. Stimulatory Effect of Loperamide on Glucose Uptake into C₂C₁₂ Myoblasts

The glucose uptake in skeletal muscle played an important role in glucose homeostasis (Baron et al., 1988; Ziel et al., 1988). Due to the short half-life of isolated skeletal muscle (Crist et al., 1988), the mouse myoblast C₂C₁₂ cell line was used in the glucose uptake experiment. After incubation with C₂Cl₁₂ Myoblasts at the cell number about 10⁶, loperamide induced an increase of 2-DG uptake with the longer of incubation time under 37° C. and reached the maximal plateau about 25 min later. Thus, glucose uptake was determined using samples incubated with loperamide for 30 min. Loparmide produced an increase of 2-DG uptake in a concentration-dependent manner from 10 nM to 10 μM. At the maximal concentration (10 μM), loperamide enhanced the glucose uptake to about 1.3 times (N=8) of the control.

In the presence of opioid μ-receptor antagonist, naloxone or naloxonazine, 2-DG uptake increased by loperamide (10 μM) was reduced. Naloxone inhibited loperamide (10 μM)-induced 2-DG uptake from 204.3±4.7 pmol/mg protein/5 min to 183.2±4.1 pmol/mg protein/5 min at 0.1 μM and to 169.9±5.0 pmol/mg protein/5 min at 1 μM in 8 experiments. Similar blockade was also observed in samples pretreated with naloxonazine at 0.1 μM and 1 μM. Thus, loperamide has the ability to enhance glucose uptake through an activation of opioid μ-receptor in C₂C₁₂ Myoblasts.

5. Effect of Loperamide on TNF-α Induced Insulin Resistance in C₂C₁₂ Myoblasts

The effect of loeramide on insulin resistance was further investigated in C₂C₁₂ myoblasts using TNF-α induced insulin resistance as described previously (Ruan et al., 2002; Mikael et al., 2002). Incubation with loperamide for 3 days at desired concentration, C₂C₁₂ myoblasts were treated with TNF-α (10 ng/ml) for one hour. Then, insulin was used to stimulate glucose uptake using 2-DG as indicator following the above method.

As shown in FIG. 8, TNF-α (10 ng/ml) induced the insulin resistance and the glucose uptake increased by insulin was markedly reduced. Incubation with loperamide reversed the glucose uptake stimulated by insulin in TNF-α treated C₂C₁₂ myoblasts in a dose-dependent manner (FIG. 8). Moreover, loperamide at maximal concentration (10 μM) reversed the glucose uptake in TNF-α induced insulin resistant C₂C₁₂ myoblasts to the level near normal control.

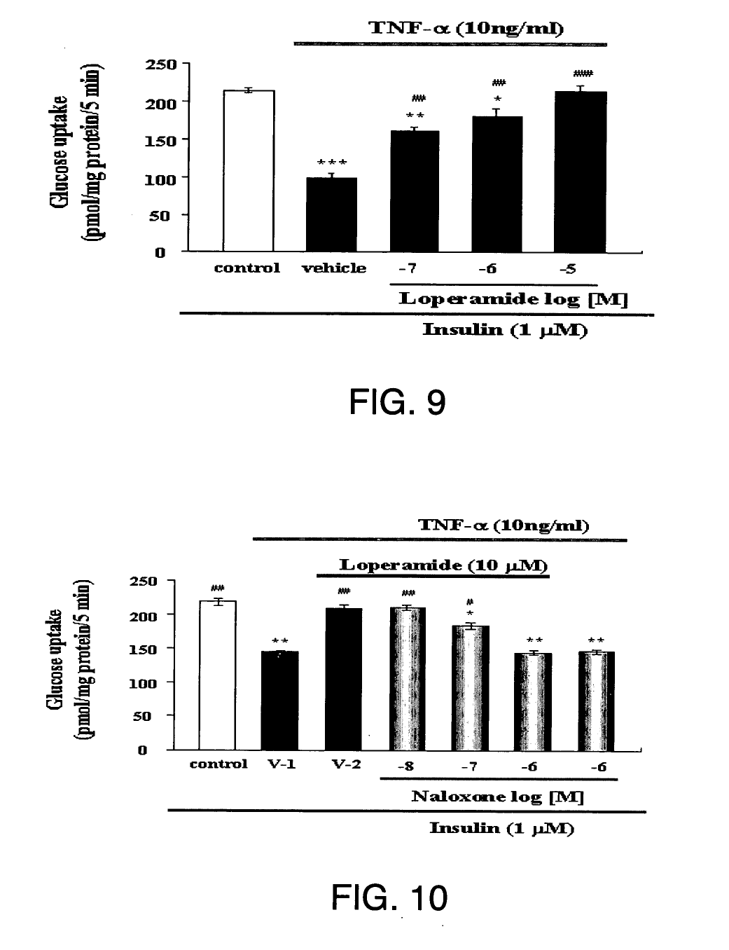

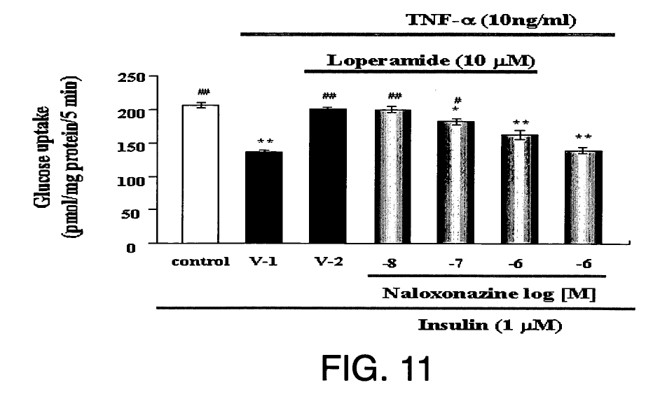

In the presence of opioid μ-receptor antagonist, naloxone or naloxonazin, actions of loperamide (10 μM) were markedly reduced (FIG. 9, FIG. 10). Naloxone or naloxonazin inhibited the effect of loperamide in a dose-dependent manner and eliminated the action of loperamide at 1 μM. Otherwise, both antagonists at 1 μM did not modify the glucose uptake stimulated by insulin in TNF-α treated C₂C₁₂ myoblasts (FIG. 9, FIG. 10).

[Discussion]

Through the results of experiments, we found that loperamide has the ability to improve insulin resistance. Using two kinds of animals with insulin resistance, loperamide was found to lower the value of glucose-insulin index in a dose-dependent manner while the value of glucose-insulin index was widely employed to evaluate insulin resistance (Kara et al., 2000; Erik et al., 2001). Also, this effect of loperamide was inhibited by naloxone at dose sufficient to block opioid receptors indicating the mediation of opioid receptors. Actually, loperamide is introduced as partial agonist of opioid μ-receptor unable to pass through blood-brain barrier. Moreover, in obese-diabetic ob/ob mice with insulin resistance, it was found to have the lowering of plasma glucose when opioid receptor was stimulated by endogenous opiate (Bailey et al., 1987). Also, insulin resistance was more easily to induce in opioid μ-receptor knock-out mice (Cheng et al., 2003). Role of opioid μ-receptor in insulin resistance can thus be considered. Therefore, activation of opioid μ-receptor by loperamide is responsible for the improvement of insulin resistance. Meanwhile, the insulin resistance improvement effect of loperamide was observed in fructose-induced insuliln resistance rats (FIG. 4) and in Zucker-diabetic fatty rats (FIG. 7); both are the well-known animal modles (Margolis et al., 1987; Wuarin et al., 1996; Saengsirisuwan et al., 2001). Hence, loperamide is effective on type II diabetes caused by food or genetic heredity. Taken together, there is no doubt that loperamide is able to improve insulin resistance.

Glucose uptake of skeletal muscle plays an important role in glucose homeostasis (Baron et al., 1988; Ziel et al., 1988). Due to the unstable of isolated skeletal muscle, we used C₂C₁₂ myoblasts of mouse myoblast cell line to investigate the effect of loperamide on glucose uptake. Loperamide was found to enhance glucose uptake in a concentration-dependent manner and this effect was inhibited by opioid μ-receptor antagonists both naloxone and naloxonazine. Thus, activation of opioid μ-receptor by loperamide is able to enhance glucose uptake into C₂C₁₂ myoblasts.

Then, we used tumor necrosis factor-α (TNF-α) to induce insulin resistance because the overexpression of TNF-α was mentioned to induce insulin resistance (Hotamisligil et al., 1993). TNF-α is one of the substances that exist in mice with malignant tumors with the ability to kill tumor cells (Carswell et al., 1975). Actually, both in vivo and in vitro, TNF-α was useful to form insulin resistance (Hotamisligil et al., 1994; Greenberg et al., 2002). Also, TNF-α is able to stimulate the secretion of leptin in fat cells and facilitate the increase of free fatty acid in blood to result in the formation of insulin resistance (Derek et al., 2001). In the cellular level, TNF-α was found to cause the phosphorylation of Ser in the insulin signals IRS-1 and IRS-2 to reduce the downstream pathway, e.g., PI 3-kinase and glucose transport protein (Hotamisligil et al., 1996). Also, TNF-α was found to cause the down-regulation of GLUT4 (Hotamisligil et al., 1993; Stephans et al., 1992 Hauner et al., 1995).

In C₂C₁₂ myoblasts, as described previously (Ruan et al., 2002; Mikael et al., 2002), TNF-α decreased the glucose uptake induced by insulin due to the formation of insulin resistance. Loperamide was found to improve the glucose uptake reduced in TNF-α induced insulin resistant cells1 (FIG. 8); wherein this effect was also inhibited by opioid α-receptor antagonists both naloxone (FIG. 9) and naloxonazine (FIG. 10). Thus, activation of opioid μ-receptor by loperamide is able to improve insulin resistance in TNF-α induced insulin resistant C₂C₁₂ myoblasts. This is further supported the view that loperamide has the ability to improve insulin resistance.

Through the activation of opioid μ-receptor, the clinical use of loperamide can be added one including ┌Improvement of insulin resistance┘.

While the present invention has been particularly shown and described with reference to a preferred embodiment, it will be understood by those skilled in the art that various changes in form and detail may be without departing from the spirit and scope of the present invention.

1. Loperamide has the ability to improve insulin resistance via activation of opioid μ-type receptor.

2. The effect of loperamide according to claim 1, wherein said this medicine is available to treat the disorders derived from insulin resistance.

3. The effect of loperamide according to claim 1, wherein said this medicine is useful for the handling of disorders caused by low function of opioid μ-receptor.
